(19) United States
(12) Patent Application Publication
(10) Pub. No.: US 2023/0050651 A1
(43) Pub. Date: Feb. 16, 2023

(54) APPARATUS, METHOD AND SET FOR THE REGENERATION OF KNEE JOINT CARTILAGES

(71) Applicant: DaThera Consumer Health GmbH, Muenchen (DE)

(72) Inventors: Christian Prinz zu Salm-Horstmar, Muenchen (DE); Alexander Schiegg, Halblech (DE)

(21) Appl. No.: 17/496,970

(22) Filed: Oct. 8, 2021

(30) Foreign Application Priority Data
Aug. 12, 2021 (DE) 102021121036.4

(51) Int. Cl. A61H 1/02; A61H 9/00

(52) U.S. Cl. CPC A61H 1/024; A61H 9/0078; A61H 2201/0192; A61H 2201/1207; A61H 2201/1642; A61H 2201/50

(57) ABSTRACT

An apparatus for the regeneration of knee joint cartilages has a support housing on which a slide part is displaceably held. The slide part has a holding and/or supporting apparatus for a leg region below a knee joint of a leg. Furthermore, a controllable movement drive moves the slide part to and fro relative to the support housing along a predefinable adjustment path. The adjustment path is predefined such that a defined compressive/tensile loading can be applied to the knee joint. The holding and/or supporting apparatus is suitable for fixing a foot in a manner free from rotation on the slide part, and/or the support housing forms a leg support which is suitable for forming a continuous supporting surface from the foot as far as a thigh region.

### CROSS-REFERENCE TO RELATED APPLICATION

This application claims the priority, under 35 U.S.C. § 119, of German Patent Application DE 10 2021 121 036.4, filed Aug. 12, 2021; the prior application is herewith incorporated by reference in its entirety.

### FIELD AND BACKGROUND OF THE INVENTION

The invention relates to an apparatus for the regeneration of knee joint cartilages according to the preamble of the independent apparatus claim.

The general view in the past was that a cartilage once damaged cannot be healed again, and a joint damaged in this way, in particular a knee joint, cannot be regenerated again. Meanwhile, there are numerous studies showing that the body is capable of healing a damaged knee joint cartilage through application of intermittent loads.

An apparatus which is suitable for this purpose having a leg support for the regeneration of knee joint cartilages is known from U.S. patent publication No. 2017/0172839 A1. This apparatus has an elongate support housing, on the supporting wall region of which a slide part with a holding apparatus for a leg region below a knee joint is held in a manner guided displaceably longitudinally. In addition, a controllable movement drive is provided between the support housing and the slide part, with which movement drive the slide part is movable to and fro over a predefinable adjustment path in relation to the support housing in order to apply a compressive/tensile loading to a knee joint having a damaged knee joint cartilage.

In the case of the known apparatus, the support housing stands on the floor in front of a chair for a patient and is connected at a relatively far distance to a seat frame of the chair by means of a linkage. As a result, the thigh of a patient above the knee joint is not supported over a relatively far region, which can be unpleasant for a patient during long term treatment and makes a therapeutically unfavorable change in position of the leg possible during a treatment.

A tensioning strap is attached to a seat element of the chair for fixing a thigh region above the knee joint, and therefore the chair with its thigh fixing here is inevitably an integral part of the apparatus. The apparatus consisting of chair and support housing is therefore large, difficult to transport and difficult to handle. This apparatus is therefore usable in practice only to a limited extent for use by a patient privately at home.

The leg region can be fixed on the slide part directly under the stretched knee joint by means of a tensioning strap and a padded part and otherwise the lower leg is exposed. The foot is merely supported in the heel region on a padded part which is adjustable in the longitudinal direction. The foot can therefore be twisted out of a therapeutically favorable, rectilinearly oriented leg position during a treatment. Such torsion has a negative effect on the regeneration of cartilage in the knee joint. Furthermore, in the event of a treatment with an excessive loading of pressure/tension on the knee joint which can be felt by a patient, the fixing on the slide part is not easily and rapidly releasable by a patient in order to rapidly end an excessive loading.

The known apparatus is preferably intended to be used for relatively short treatment times of on average six minutes per day in a clinic.

### SUMMARY OF THE INVENTION

It is the object of the invention to propose an apparatus which can be produced simply and compactly and can be handled simply, in particular even can be handled by a patient at home, by means of which an optimum regeneration of knee joint cartilages can be carried out.

This object is achieved by the features of the independent claims. Advantageous refinements are the subject matter of the dependent claims which are referred back thereto.

According to the independent claim, an apparatus for the regeneration of knee joint cartilages is provided, which has a, preferably elongate, support housing on which a slide part is displaceably held, wherein the slide part has a holding and/or supporting apparatus for a leg region below a knee joint of a leg. Furthermore, the apparatus has a controllable movement drive by means of which the slide part is movable to and fro relative to the support housing along a predefinable adjustment path. The adjustment path is predefined in such a manner that a defined compressive/tensile loading can be applied to the knee joint of a leg resting and/or held on the slide part.

According to a first aspect of the invention, it is provided that the holding and/or supporting apparatus is suitable for fixing at least the foot, preferably the foot and the adjoining lower leg or calf region, in a manner free from rotation on the slide part. Therefore, at least the foot is advantageously held stably for the entire treatment time in the desired position set at the start of the treatment, which has a positive influence on regeneration, in particular in the event of more lengthy treatment periods.

According to a second aspect of the invention which can be provided additionally or alternatively to the first aspect of the invention, it is provided that the support housing forms or has a, preferably plate-like, leg support which is suitable for forming a continuous supporting surface from the foot as far as a thigh region, adjoining the knee joint, of a leg, wherein the slide part forms part of the leg support and, with a clearance gap which has at least a width corresponding to the movement of the slide part along the predefinable adjustment path, adjoins a partial leg supporting region formed or arranged in a manner mounted on the support housing. As a result, the leg rests in the lower region fully supported over its length on the leg support.

With the solution according to the invention according to the first and/or the second aspect of the invention, a leg having a damaged knee joint cartilage is therefore advantageously held stably for the entire treatment period in the leg position desired and set at the start of the treatment, which has a positive effect on regeneration, in particular in the event of more lengthy treatment periods.

For the fixing in a manner free from rotation, according to a particularly preferred refinement, at least one inflatable apparatus can be provided which, in the inflated state, fixes at least the foot, preferably the foot and the adjoining lower leg or calf region, in a manner free from rotation on the slide part. Such an inflatable apparatus permits particularly simple, functionally secure, individually adjustable and reliable rotation-free fixing with the possibility, if desired, of the holding force being distributed over a large surface area, which makes the treatment particularly pain-free and comfortable for a patient.

According to a further particularly preferred specific refinement, the holding and/or supporting apparatus can be formed by a foot housing arranged or formed on the support housing or can have such a foot housing which permits simple and comfortable accommodating and mounting of a foot, in particular if the foot housing is formed with a substantially U-shaped cross section so as to be open upward and to the rear in the direction of the patient. The foot and optionally calf region can be rapidly and simply inserted into, and also pulled out again from, such a foot housing.

The possibility of being able to always rapidly and simply pull out the foot also signifies protection against an excessive action force and thus possibly associated pain for the patient being treated. Also for this reason, inter alia, an apparatus is advantageous in which the holding and/or supporting apparatus, preferably the inflatable apparatus, in its use position fixes a foot or a foot together with an adjoining lower leg or calf region with a predefined holding force, preferably with a holding force which is adjustable by inflation of the inflatable apparatus. This is because, with such a construction, the holding force can then be predefined in a particularly simple manner such that, after the holding force is exceeded, the foot can be extracted from the holding and/or supporting apparatus, preferably from the inflatable apparatus, for example can simply be pulled out of the foot housing which is formed open upward and to the rear in the direction of the patient. This variant expressly also contains the possibility that the foot, when it is subjected to too much tension, will automatically slide out of the holding and/or supporting apparatus, preferably out of the inflatable apparatus.

A construction can be produced particularly simply in conjunction with a foot housing, in which the foot housing forms or has the at least one inflatable inner region as the inflatable apparatus. A particularly functionally secure and reliable rotation-free fixing is provided here with a construction in which the foot housing forms or has two inflatable inner side regions lying on opposite sides as the inflatable apparatus.

According to a further particularly preferred refinement, a shell part, which is preferably manufactured from a plastics material, can be inserted into the foot housing, the shell part having or forming the at least one inner region and thus the at least one inflatable apparatus. Such a shell part permits particularly simple and individual adaptation to different foot and leg shapes or sizes. The shell part can be held exchangeably on the foot housing in order, for example, to be able to provide a set of shell parts in different sizes. Accordingly, it is preferably provided that the apparatus has a plurality of shell parts, which form a shell part set, in different sizes at least with respect to an interior space receiving the foot. For example, the interior spaces of the individual shell parts can be kept in reserve in small, medium, large and extra-large sizes. An even more individual adaptation to different patient conditions is thus possible. The shell part is particularly advantageously manufactured here from a plastics material thus promoting a lighter, less heavy construction. In principle, however, the shell part can, of course, also be formed integrally with the foot housing.

The shell part is preferably configured with a large-volume interior space which can be constricted to an individual foot shape and calf shape by the inflatable apparatus for holding and rotation-free fixing. The shell part can therefore be configured with an interior space corresponding to a very large foot and calf shape. The apparatus is thus universally usable for different lengths and leg and foot shapes of patients. For example, the shell part can also be longer here than the foot housing, as seen in the longitudinal direction and/or in the vertical axis direction of the leg support.

A simple construction in terms of production is produced especially if the shell part is substantially adapted the shape of the foot housing.

Particularly comfortable insertion and removal of the foot is produced with a shell part which is formed with a substantially U-shaped cross section so as to be open upward and to the rear in the direction of the patient.

For functionally secure and reliable fixing, it is advantageous if the inflatable apparatus has at least one inflatable cushion which is provided, preferably integrated, in the at least one inner region, or is formed by such a cushion, said cushion, in the inflated state, fixing the foot and optionally the adjoining calf region in a manner free from rotation. A specific embodiment is particularly advantageous here in which the inflatable apparatus in each case has at least one inflatable cushion which is provided, preferably integrated, in the opposite inner side regions, the cushions, in the inflated state, fixing the foot and optionally the adjoining calf region in a manner free from rotation, specifically laterally enclosing same and fixing them in a manner free from rotation.

The at least one inflatable cushion can be connected, for example through a wall of the foot housing or shell part, to a pump device via a tube. For this purpose, a respective inflatable, bellows-shaped cushion can advantageously be inserted into a respective side region of the shell part, the cushions each being connected through the shell wall of the shell part to a pump via a tube. The pump device or pump can be arranged, for example, on a housing region of the support housing which, preferably in the use position, is assigned to the knee or thigh region of a patient, and is thus easily accessible, or, however, alternatively can also be formed by a separately handleable pump which is connected to the respective inflatable cushion via a tube, wherein the tube is preferably of such a length that the pump can be operated by the patient in an ergonomic sitting position. In both cases mentioned, a suitable pump device or pump is a manually actuable pump, in particular a manually actuable rubber pump. Rapid and comfortable fixing of the foot and of a calf part is therefore possible by a patient themselves without outside support. Alternatively, however, a pump device which is activatable by means of a control device, for example by means of a manual control unit, can be provided which, for example, is integrated in the support housing or in the foot housing or in the shell part and is connected to the inflatable apparatus, preferably to the at least one inflatable cushion, for example via tube lines. The activation can be undertaken here, for example, in a remote controlled or wired manner, to mention only one exemplary embodiment.

The foot housing is particularly preferably formed in a dimensionally stable manner such that an overall stable construction is produced which assists a reliable holding of the foot in the foot housing. The foot housing can be formed here in the manner of an overshoe.

The foot housing can also be formed integrally with the slide part or can be formed by a separately handleable component.

The apparatus itself is preferably integral, relatively small and compact, can be easily stowed away and is readily usable for use at home.

For individual length adaptation to a leg length of a current patient, the foot housing is connected to the slide part via a longitudinal adjustment device. Such a longitudinal adjustment can be realized by a multiplicity of different and generally known adjustment elements, in particular, for example, via perforated tubes or perforated rails with adjustment sections corresponding to the distances between the holes or for continuous adjustments with guides and clamping apparatuses.

A cost-effective, simple and functional embodiment is obtained with a foot housing of metal sheet in the manner of a box-shaped component, for example also in conjunction with a shell part which is longer by comparison and is made from a plastics material.

According to a further particularly preferred, specific refinement, the slide part and the partial leg supporting region mounted on the support housing form a leg support which, as seen in the vertical axis direction, is arranged in an upper region of the support housing, preferably is arranged there flush with the surface or substantially flush with the surface. Such a leg support can be produced simply and permits a comfortable, continuous leg support from the foot as far as the thigh region adjoining the knee joint.

According to a further particularly preferred refinement, the partial leg supporting region mounted on the support housing protrudes from the support housing in the manner of a plate and/or as seen in the longitudinal direction of the support housing, wherein it forms a supporting and support device, preferably for supporting and support on a seat surface edge of an item of seating furniture. For example, in the operating position, the apparatus can be supported in an oblique position downward with the opposite front housing lower edge on the floor or on a comparable support, or, alternatively thereto, in the operating position, the apparatus can be arranged in a manner oriented horizontally or substantially horizontally, depending on which sitting posture is more comfortable for a patient.

Furthermore, the end-side region of the partial leg supporting region can have a defined supporting portion which is suitable in particular to be placed and/or supported on a seat surface edge.

The supporting portion can be formed, for example, in a manner such that it can be bent or angled on the partial leg supporting region, for example can be angled via a releasable and re-fixable joint device, or alternatively can be angled by a configuration of the supporting portion which is flexible at least in partial regions. With such a design which can be bent or angled, the apparatus can be used both in an obliquely positioned manner and horizontally, depending on the patient's requirements, and can nevertheless rest and be supported in a functionally secure and reliable manner on the seat surface edge, for example.

In principle, a multiplicity of motor variants and structural possibilities known per se are available as the movement drive. A particularly simple, specific and functional embodiment is obtained with a movement drive which is designed as a pneumatic and/or hydraulic and/or electric drive.

A particularly preferred, functionally reliable movement drive which is less prone to faults is a spindle drive, in which the spindle motor is attached to the slide part, preferably is attached from below to the slide part, as seen in the vertical axis direction, wherein a spindle, which is rotationally drivable by the spindle motor, of the spindle drive is supported in a spindle bearing mounted on the support housing. For example, the spindle bearing can be formed by a fixed transverse wall of the support housing, the transverse wall having a spindle nut.

In addition, for the functionally secure and reliable guiding and holding of the slide part, a slotted guide mechanism can be formed on the support housing, in and/or on which the slide part is guided in a longitudinally displaceable manner.

Specifically, it can be provided here, according to a first exemplary embodiment, that the slotted guide mechanism has, on opposite sides of the support housing in the transverse direction, a respective guide rail as lift-off protection, the guide rails, preferably on their lower side, as seen in the vertical axis direction, each being assigned a plurality of rollers which are spaced apart in the adjustment direction of the slide part and are arranged mounted on the housing such that, for example, in each case an edge region of the slide part is accommodated, in a substantially form-fitting manner, between the guide rails and the rollers, and, during an adjustment along the predefined adjustment path, the slide part rolls and is supported on the rollers.

Alternatively thereto, it can be provided, according to a further preferred embodiment, that the slotted guide mechanism has, on opposite sides of the support housing in the transverse direction, in each case a plurality of rollers which are spaced apart in the adjustment direction of the slide part and are arranged mounted on the housing. In addition, the slotted guide mechanism furthermore has, on the opposite edge regions of the slide part, the edge regions being assigned to the rollers, at least one cross-sectionally U-shaped roller receptacle which receives the rollers such that the slide part rolls and is supported on the rollers during an adjustment along the predefined adjustment path. It goes without saying that, in the case of this variant embodiment, the kinematic reversal, in which the rollers are arranged on the slide part and the U-shaped roller receptacles on the support housing, is of course also covered by the scope of protection.

Furthermore alternatively, a pure sliding guide is also conceivable in which the slotted guide mechanism, on opposite sides of the support housing in the transverse direction, in each case has at least one cross-sectionally U-shaped receptacle in which an associated edge region of the slide part is accommodated so as to be guided in a longitudinally displaceable manner, preferably is accommodated in a substantially form-fitting manner. It goes without saying that, also in the case of this variant embodiment, the kinematic reversal, in which the U-shaped receptacles are arranged on the slide part while the edge regions engaging there are formed or arranged on the support housing, is of course again covered by the scope of protection.

For the starting and for the controlling of the apparatus, the latter advantageously has a control device, preferably a wired handheld operating unit as the control device, wherein the control device is connected to the movement drive, preferably for the controlled adjustment of the slide part along an adjustment path corresponding to a sinusoidal traction movement, in which the knee is subjected to a repeated compressive/tensile loading. In addition, the movement drive can preferably be activated by means of the control device in such a manner that an adjustment path and/or an adjustment speed and/or an adjustment sequence and/or a running time and/or the size of a transmissible force and/or an interruption in the movement can be set.

The controller here is configured for a particularly advantageous regeneration of the knee joint cartilage up to a maximum adjustment path of 10 cm, preferably of 8 cm, at most preferably of 6 cm, and/or up to a maximum adjustment speed of 8 mm per second, preferably of 6 mm per second, most preferably of 4 mm per second, preferably as a sinusoidal movement, and/or for a maximally transmissible force of 360 N. The treatment duration is, for example, between 1 hour and 8 hours per day. With treatments within the ranges, good treatment success can be achieved while avoiding overloading.

The apparatus is suitable in particular for long term therapy of several weeks in accordance with the stipulations of a doctor.

The advantages produced with the method correspond to those of the previously acknowledged apparatus.

Furthermore, a set is also claimed with which the previously mentioned advantages are likewise produced.

In addition, it goes without saying that the apparatus according to the invention, the method according to the invention and the set according to the invention together with their above-explained advantageous developments are of course equally suitable for use on a left and right foot/leg.

Other features which are considered as characteristic for the invention are set forth in the appended claims.

Although the invention is illustrated and described herein as embodied in an apparatus for the regeneration of knee joint cartilages, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a diagrammatic, perspective view of an apparatus with a leg support for the regeneration of knee joint cartilages according to the invention;

FIG. 2 is a longitudinal sectional view taken along the line II-II shown in FIG. 3 of the apparatus according to FIG. 1;

FIG. 2A is a sectional view of a slotted guide mechanism in the embodiment of a sliding guide;

FIG. 3 is a bottom plan view of the apparatus according to FIG. 1;

FIG. 4 is a top plan view of the apparatus into a foot housing with a shell part;

FIG. 5 is an enlarged partial, perspective view according to FIG. 4 with an inserted and fixed foot; and

FIG. 6 is a perspective view of the apparatus according to FIG. 1 in its functional position with a patient.

### DETAILED DESCRIPTION OF THE INVENTION

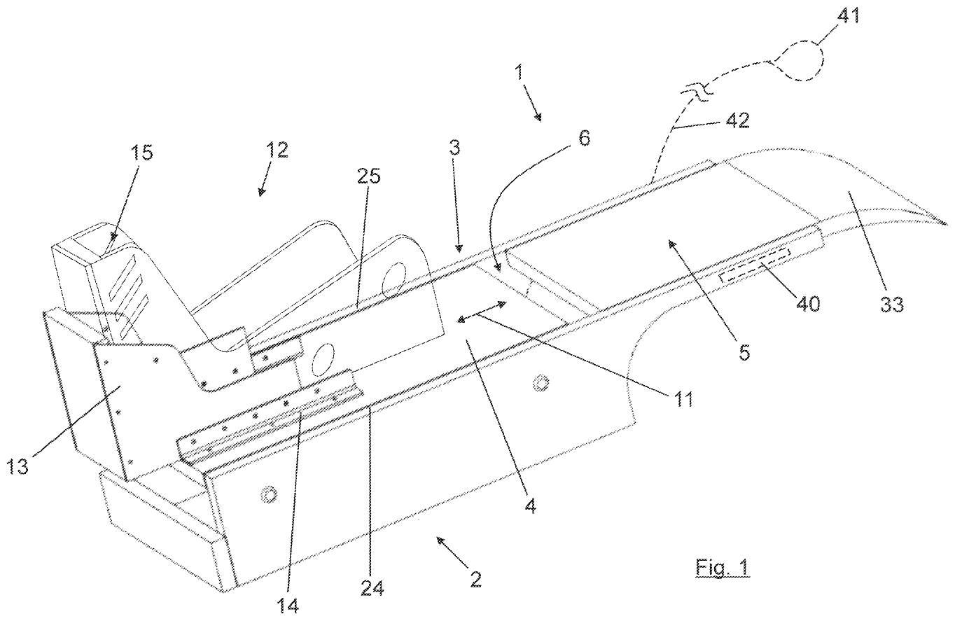

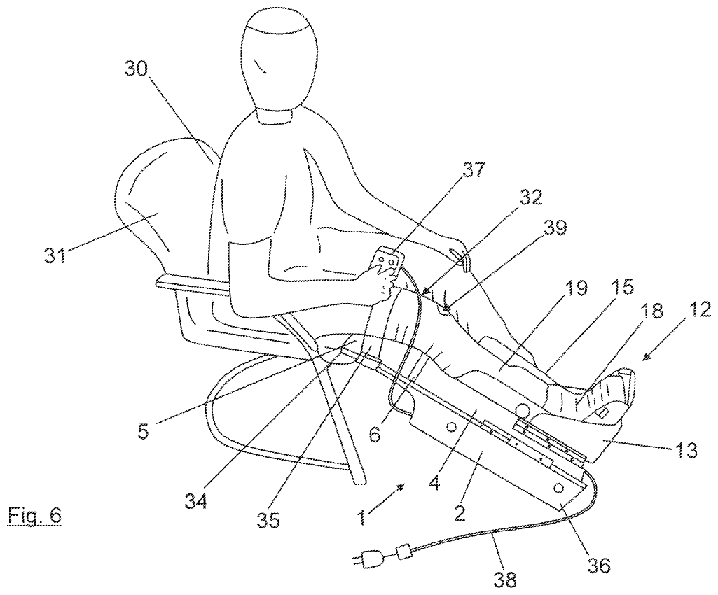

Referring now to the figures of the drawings in detail and first, particularly to FIG. 1 thereof, there is shown an apparatus 1 for the regeneration of knee joint cartilages. The apparatus 1 contains an elongate support housing 2 with a leg support 3 which forms a continuous supporting surface from a foot 18 as far as a thigh region, adjoining a knee joint 39, of a patient 30 (also see FIG. 6).

A slide part 4 forms part of the plate-like leg support 3, which is formed here flush with the surface merely by way of example, wherein a partial leg supporting region 5, which is arranged mounted on the support housing, adjoins the slide part 4 with a clearance gap 6.

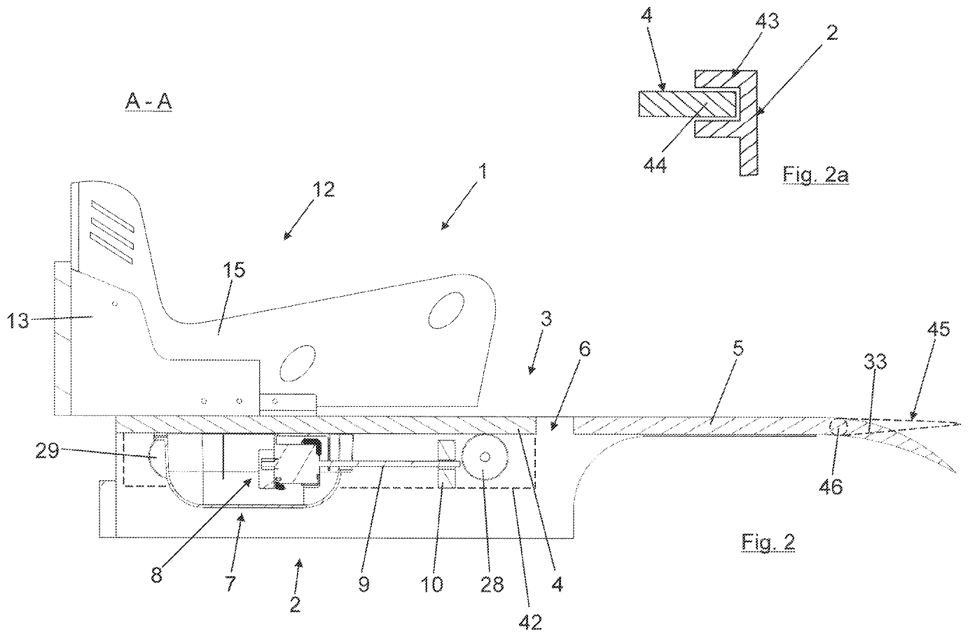

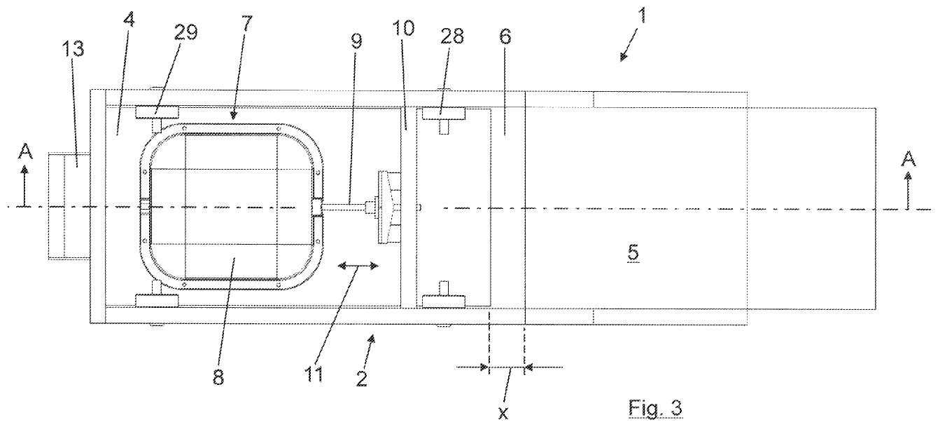

A movement drive 7 (see FIG. 2) is firstly attached from below to the slide part 4 in the support housing 2, which is open downward here, as an, for example, electric spindle drive 8 and secondly is supported with its spindle 9 on a transverse wall 10 of the support housing 2. The clearance gap 6 has a width which permits a movement of the slide part 4 along a predefinable adjustment path. The adjustment path x is shown merely schematically and by way of example in FIG. 3 as corresponding to the width of the clearance gap, but can or should in practice be slightly smaller than the width of the clearance gap.

The slide part 4 can be moved to and fro, by means of the movement drive 7, in the longitudinal direction, corresponding to the arrow 11, relative to the support housing 2, which is arranged or held in a positionally fixed manner, in conjunction with a control by means of a control device, specifically preferably with a sinusoidal movement sequence, wherein optionally the adjustment speed, the adjustment path and further parameters can be adjusted, preferably according to the stipulations of a doctor.

A holding and/or supporting apparatus 12 for a leg region below the knee joint 39 of a patient 30 is arranged on the slide part 4. The holding and/or supporting apparatus 12 here has, by way of example, a preferably dimensionally stable foot housing 13 similar to an overshoe which here furthermore by way of example is open to the rear in the direction of the patient and upward.

The foot housing 13 here furthermore by way of example is manufactured as a box-shaped component from, for example, metal sheet and is connected, preferably releasably, to the slide part 4. In principle, however, a connection which is not destruction-free could also be provided in the same way as an integral design.

For an individual adaptation of the length to a patient, the connection is produced via a longitudinal adjustment device, illustrated here merely schematically and by way of example by an adjustable perforated rail arrangement 14.

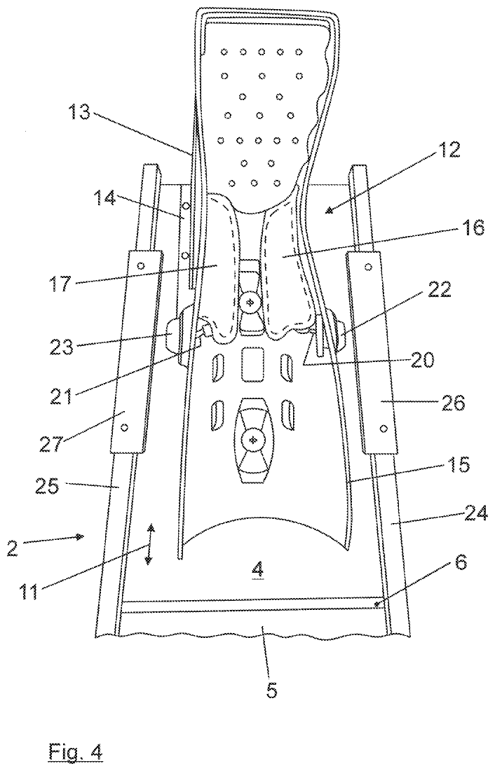

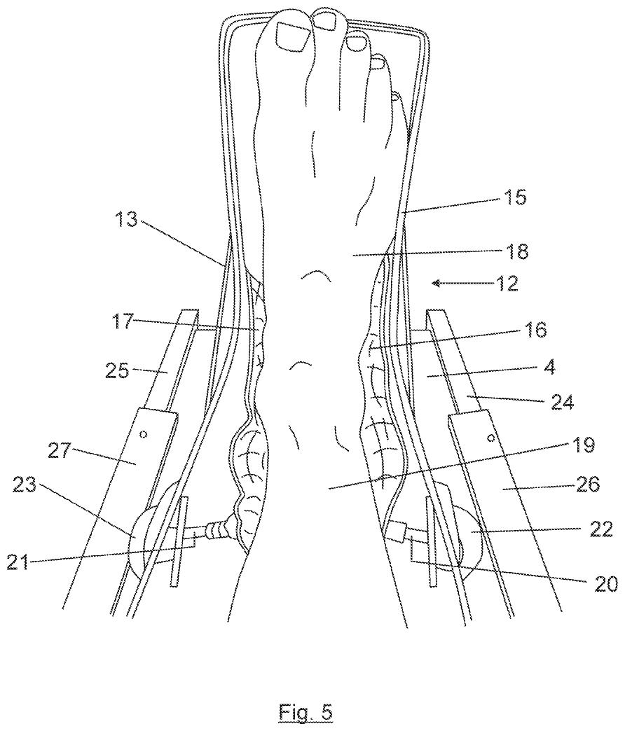

Inserted into the foot housing 13 here is a shell part 15 which is adapted in shape to the interior thereof, is likewise open to the rear and upward and in which inflatable cushions 16, 17 are attached in inner opposite side regions (see FIG. 4), the cushions, in the inflated state, fixing a foot 18 inserted in the shell part 15 and optionally (as shown here by way of example) laterally enclosing and fixing, in a manner free from rotation, an adjoining lower leg region or calf region 19 from below (see FIG. 5).

For this purpose, the shell part 15 which is preferably longer than the foot housing 13 can be produced, for example, from a plastics material with an interior space which corresponds to a large foot and calf shape and which can be constricted to an individual foot shape and calf shape by the inflated cushions 16, 17 for holding and fixing a leg in a manner free from rotation.

In order to form a set, the shell part 15 can be held exchangeably on the foot housing 13 and a plurality of shell parts 15 forming a shell part set can be stored or present in different sizes, at least with respect to an interior space receiving the foot 18, but this is not illustrated here.

The inflatable, preferably bellows-shaped cushions 16, 17 in the interior side region of the shell part 15 are connected here merely by way of example and for better clarification in each case through the shell wall in a flow-conducting manner via a tube 20, 21 to a respectively assigned pump which is attached to the outer side of the foot housing and here is a manually actuable rubber pump 22, 23 (see FIGS. 4 and 5). The pumps 22, 23 are arranged here on the outer side of the foot housing merely in order to explain the basic concept of the invention. However, an arrangement of the pumps that is ergonomically more favorable for the patient, for example in a housing region of the support housing 2 that is assigned to the knee region or thigh region of the patient, is particularly preferred, this being illustrated here merely extremely schematically by the pump 40 in dashed line in FIG. 1. Alternatively, an embodiment which is likewise illustrated in FIG. 1 is also conceivable, in which the pump device is formed by a separately handleable pump 41 which is connected to the respective inflatable cushion via a tube 42, wherein the tube then preferably is of such a length that the pump can be operated by the patient in an ergonomic sitting position.

For longitudinally displaceably guiding and holding the slide part 4, a slotted guide mechanism is formed on the support housing 2, which is apparent in particular from FIGS. 1, 2, 4 and 5:

The transverse guide for the slide part 4 is realized here merely by way of example by opposite side walls or sides 24, 25 of the support housing 2 which run in the vertical axis direction and on which, here merely by way of example, guide rails 26, 27 are arranged, as lift-off protection, in the manner of plate parts (omitted in FIGS. 1 and 2), which engage over the slide part, as part of the slotted guide mechanism. The plate-like slide part 4 is downwardly supported, for a smooth rolling movement, on at least two rollers 28, 29, which are each arranged on both sides in a manner mounted on the housing and likewise form part of the slotted guide mechanism (see FIG. 2).

Alternatively, the slotted guide mechanism can have, on opposite sides 24, 25 of the support housing 2 in the transverse direction, in each case, as shown in FIG. 2, a plurality of rollers 28, 29 which are spaced apart in the adjustment direction of the slide part 4, are arranged mounted on the housing and are accommodated in cross-sectionally U-shaped roller receptacles 42 which are assigned to the rollers 28, 29 on opposite edge regions of the slide part 4 such that the slide part 4 rolls and is supported on the rollers 28, 29 during an adjustment along the predefined adjustment path. FIG. 2 shows one such U-shaped roller receptacle 42, which is designed for example as a U rail, merely in extremely schematic form by dashed lines.

FIG. 2A also shows merely extremely schematically and by way of example a third embodiment of the slotted guide mechanism, in which the slotted guide mechanism is configured as a sliding guide and, on opposite sides of the support housing 2 in the transverse direction, in each case has at least one cross-sectionally U-shaped receptacle 43 in which an associated edge region 44 of the slide part 4 is accommodated in a substantially form-fitting manner and so as to be guided in a longitudinally displaceable manner.

FIG. 6 shows the apparatus 1 in its functional and operating position in conjunction with a patient 30 in an ergonomically favorable sitting position. The patient 30 is sitting here on a chair 31 and has here, by way of example, their right leg 32 together with their foot 18 and the adjoining lower leg or calf region 19 already fixed in the shell part 15 in a manner free from rotation by the inflated cushions 16, 17.

The partial leg support region 5, which is mounted on the housing, of the leg support 3 protrudes here upward as a plate part on the support housing 2 and rests with a supporting portion 33, which is slightly bent downward, as seen in the vertical axis direction, on a front seat surface edge 34 of a chair 31 and is supported there. The thigh region above the knee joint 39 and/or the position of the support wall part 5 on the seat surface edge 34 can optionally also be secured in position here by a tensioning strap 35.

The supporting portion 33 is formed flexibly here, for example at least in the bent partial region, in order to be able to transfer the supporting portion 33, for example in conjunction with horizontal use of the apparatus 1, into the horizontal position 45 shown by dashed lines in FIG. 1. This takes place, for example, by suitable selection of material or alternatively also by the provision of a joint 46.

The apparatus 1 with its leg support is tilted obliquely forward here merely by way of example and is supported with a front housing lower edge 36 on the floor in front of the patient 30. The patient 30 therefore sits in a relaxed state and comfortably with a slightly angled leg 32 during a treatment, wherein this position in conjunction with the rotation-free fixing of the leg promotes a successful treatment.

Also connected to the movement drive 7 is a hand control unit 37 which the patient 30 here holds in the hand and with which movement parameters are fixedly or variably predefinable. In addition, a network cable 38 for the energy supply of the movement drive 7 is shown schematically. The hand control unit 37 can optionally also activate and actuate a pump device which is integrated, for example, in the supporting housing 2 or in the foot housing 13 or in the shell part 15 and is connected to the at least one inflatable cushion, which is, however, not illustrated here. The activation here can take place, for example, in a remote-controlled or wired manner, to mention only one exemplary embodiment.

During use, the patient 30 uses the apparatus with the leg support, for example, at home according to the patient's doctor's instructions. The mobile apparatus 1 can be positioned and used in front of any chair 31, sofa or couch. The patient 30 positions and fixes the relevant leg 32 on the leg support and starts the apparatus 1 which now, with the traction prescribed by the doctor, draws the knee 39 apart and returns it again along a sinusoidal function, wherein the knee 39 is drawn apart in a recurring cycle and is then relieved of load again. The treatment duration is stipulated by the doctor, wherein the starting point can be a minimum of approximately 1 hour per day.

The following is a summary list of reference numerals and the corresponding structure used in the above description of the invention:
1 Apparatus
2 Support housing
3 Leg support
4 Slide part
5 Partial leg supporting region mounted on the housing
6 Clearance gap
7 Movement drive
8 Spindle drive
9 Spindle
10 Transverse wall
11 Arrow
12 Holding and/or supporting apparatus
13 Foot housing
14 Perforated rail
15 Shell part
16 Inflatable cushion
17 Inflatable cushion
18 Foot
19 Lower leg or calf region
20 Tube
21 Tube
22 Rubber pump
23 Rubber pump
24 Side
25 Side
26 Guide rail
27 Guide rail
28 Roller
29 Roller
30 Patient
31 Chair
32 Leg
33 Supporting portion
34 Seat surface edge
35 Tensioning strap
36 Housing lower edge
37 Hand control unit
38 Network cable
39 Knee joint
40 Pump
41 Pump
42 Tube
43 U-shaped receptacle
44 Edge region
45 Horizontal position

1. An apparatus for regeneration of knee joint cartilages, the apparatus comprising:
a slide part having a holding and/or supporting apparatus for a leg region below a knee joint of a leg;
a support housing on which said slide part being displaceably held;
a controllable movement drive by which said slide part being movable to and fro relative to said support housing along a predefinable adjustment path, the predefinable adjustment path being predefined in such a manner that a defined compressive/tensile loading can be applied to the knee joint of the leg resting and/or held on said slide part;
said holding and/or supporting apparatus being suitable for fixing at least a foot in a manner free from rotation on said slide part, and/or
in that said support housing forming or having a leg support being suitable for forming a continuous supporting surface from the foot as far as a thigh region, adjoining the knee joint, of the leg;
a partial leg supporting region; and
said slide part forming part of said leg support and, with a clearance gap which has at least a width corresponding to a movement of said slide part along the predefinable adjustment path, adjoins said partial leg supporting region formed or disposed in a manner mounted on said support housing.

2. The apparatus according to claim 1, wherein said holding and/or supporting apparatus has at least one inflatable apparatus which, in an inflated state, fixes at least the foot in a manner free from rotation on said slide part.

3. The device according to claim 2, wherein said holding and/or supporting apparatus is formed by a foot housing disposed or formed on said support housing.

4. The apparatus according to claim 1, wherein said holding and/or supporting apparatus in its use position fixes the foot or the foot together with an adjoining lower leg or calf region with a predefined holding force.

5. The apparatus according to claim 3, wherein said foot housing has at least one inflatable inner region as said at least one inflatable apparatus, wherein said at least one inflatable inner region is suitable, in the inflated state, for fixing at least the foot in a manner free from rotation.

6. The apparatus according to claim 5, further comprising a shell part being inserted into said foot housing, said shell part having or forming said at least one inflatable inner region and thus said at least one inflatable apparatus.

7. The apparatus according to claim 6, wherein said shell part is held exchangeably on said foot housing.

8. The apparatus according to claim 6, wherein said shell part is configured with an interior space which can be constricted to an individual foot shape and calf shape by said at least one inflatable apparatus for holding and fixing the leg in a manner free from rotation, wherein said shell part is longer than said foot housing, as seen in a longitudinal direction and/or in a vertical axis direction of said leg support.

9. The apparatus according to claim 6, wherein said shell part is substantially adapted to a shape of said foot housing, and/or in that said shell part is formed with a substantially U-shaped cross section so as to be open upward and to a rear in a direction of a patient.

10. The apparatus according to claim 5, wherein said at least one inflatable apparatus has at least one inflatable cushion which is provided in said at least one inflatable inner region and, in the inflated state, fixes the foot and optionally an adjoining calf region in a manner free from rotation.

11. The apparatus according to claim 10, wherein said at least one inflatable apparatus is one of a plurality of inflatable cushions which are provided in opposite inner side regions, said inflatable cushions, in the inflated state, fixing the foot and optionally the adjoining calf region in a manner free from rotation.

12. The apparatus according to claim 10,
further comprising a pump;
further comprising a tube; and
wherein said inflatable cushions are connected to said pump via said tube, wherein said pump is disposed on a housing region of said support housing which is assigned to the knee or thigh region of a patient, or said pump is formed by a separately handlable pump which is connected to a respective one of said inflatable cushions via said tube.

13. The apparatus according to claim 3, wherein said foot housing is formed in a dimensionally stable manner and/or in a manner of an overshoe.

14. The apparatus according to claim 3, wherein said foot housing is formed integrally with said slide part or is formed by a separately handlable component.

15. The apparatus according to claim 3, further comprising a longitudinal adjustment device, said foot housing being connected to said slide part for individual length adaptation via said longitudinal adjustment device.

16. The apparatus according to claim 3, wherein said foot housing is a box-shaped component produced from metal sheet.

17. The apparatus according to claim 1, wherein said slide part and said partial leg supporting region mounted on said support housing form said leg support which, as seen in a vertical axis direction, is disposed in an upper region of said support housing.

18. The apparatus according to claim 1, wherein:
said partial leg supporting region mounted on said support housing protrudes from said support housing in a manner of a plate and/or as seen in a longitudinal direction of said support housing and forms a supporting and support device; and
in an operating position, the apparatus can be supported in an oblique position downward with an opposite front housing lower edge on a floor, or that, in the operating position, the apparatus is disposed in a manner oriented horizontally or substantially horizontally.

19. The apparatus according to claim 1, wherein said partial leg supporting region is equipped with a fixing device.

20. The apparatus according to claim 18, wherein said partial leg supporting region has an end-side region with a supporting portion.

21. The apparatus according to claim 1, wherein said controllable movement drive is a pneumatic and/or hydraulic and/or electric drive.

22. The apparatus according to claim 21,
further comprising a spindle bearing mounted on said support housing; and
wherein said controllable movement drive is a spindle drive having a spindle motor attached to said slide part as seen in a vertical axis direction, said spindle drive further has a spindle, which is rotationally drivable by said spindle motor and is supported in said spindle bearing mounted on said support housing.

23. The apparatus according to claim 1, wherein said support housing has a slotted guide mechanism formed thereon, and in and/or on said slotted guide mechanism said slide part is guided in a longitudinally displaceable manner for guiding and for holding said slide part.

24. The apparatus according to claim 23, wherein said slotted guide mechanism has, on opposite sides of said support housing in a transverse direction, a respective guide rail of guide rails as lift-off protection, said guide rails, as seen in a vertical axis direction, each being assigned a plurality of rollers which are spaced apart in an adjustment direction of said slide part and are disposed mounted on said support housing, and in that in each case an edge region of said slide part is accommodated, in a substantially form-fitting manner, between said guide rails and said rollers such that, during an adjustment along the predefined adjustment path, said slide part rolls and is supported on said rollers.

25. The apparatus according to claim 23, wherein said slotted guide mechanism has, on opposite sides of said support housing in a transverse direction, in each case a plurality of rollers which are spaced apart in an adjustment direction of said slide part and are disposed mounted on said support housing, and in that said slotted guide mechanism furthermore has, on opposite edge regions of said slide part, said opposite edge regions being assigned to said rollers, at least one cross-sectionally U-shaped roller receptacle which receives said rollers such that said slide part rolls and is supported on said rollers during an adjustment along the predefined adjustment path.

26. The apparatus according to claim 23, wherein said slotted guide mechanism is configured as a sliding guide and, on opposite sides of said support housing in a transverse direction, in each case has at least one cross-sectionally U-shaped receptacle in which an associated edge region of said slide part is accommodated in a manner so as to be guided in a longitudinally displaceable manner.

27. The apparatus according to claim 1, further comprising a controller connected to said controllable movement drive for a controlled adjustment of said slide part along an adjustment path corresponding to a sinusoidal traction movement, in which the knee is subjected to a repeated compressive/tensile loading.

28. The apparatus according to claim 27, wherein said controllable movement drive can be activated by means of said controller in such a manner that an adjustment path and/or an adjustment speed and/or an adjustment sequence and/or a running time and/or a size of a transmissible force and/or an interruption in a movement can be set.

29. The apparatus according to claim 28, wherein said controller is configured for controlling said controllable movement drive up to a maximum adjustment path of 10 cm, and/or up to a maximum adjustment speed of 8 mm per second and/or for a maximally transmissible force of 360 N.

30. A method for operating the apparatus according to claim 1 and the apparatus further having a controller connected to the controllable movement drive, which comprises the steps of:
operating the controller which is connected to the controllable movement drive for a controlled adjustment of the slide part along the predefinable adjustment path corresponding to a sinusoidal traction movement, in which the knee is subjected to a repeated compressive/tensile loading.

31. A set, comprising:
an apparatus for a regeneration of knee joint cartilages, said apparatus containing:
a slide part having a holding and/or supporting apparatus for a leg region below a knee joint of a leg;
a support housing on which said slide part is displaceably held;
a controllable movement drive, by means of said controllable movement drive said slide part is movable to and fro relative to said support housing along a predefinable adjustment path, wherein the predefinable adjustment path is predefined in such a manner that a defined compressive/tensile loading can be applied to the knee joint of the leg resting and/or held on said slide part;
said holding and/or supporting apparatus is suitable for fixing at least a foot in a manner free from rotation on said slide part, and is formed by a foot housing disposed or formed on said support housing; and
a shell part inserted into said foot housing, said shell part having or forming at least one inner region and thus at least one inflatable device, wherein said shell part being held exchangeably on said foot housing, and wherein said shell part of said apparatus is one of a plurality of shell parts, which form a shell part set, in different sizes at least with respect to an interior space receiving the foot.
